(19) United States
(12) Patent Application Publication
(10) Pub. No.: US 2009/0304756 A1
(43) Pub. Date: Dec. 10, 2009

(54) Method for the Encapsulation and Controlled Release of Poorly Water-Soluble (Hyprophobic) Liquid and Solid Active Ingredients

(76) Inventors: Lars Dähne, Berlin (DE); Steffi Senst, Potsdam (DE); Barbara Baude, Schwielowsee (DE); Andreas Voigt, Berlin (DE)

Correspondence Address: Leon R. Yankwich;Yankwich & Associates, 201 Broadway, Cambridge, MA, 02139, US

(21) Appl. No.: 11/992,083

(22) PCT Filed: Sep. 18, 2006
(86) PCT No.: PCT/EP2006/009063

§ 371 Date: Jul. 7, 2009

(30) Foreign Application Priority Data
Sep. 16, 2005 (DE) 10 2005 044 400.8

(51) Int. Cl. A61K 8/02; B05D 3/00; A61K 9/14

(52) U.S. Cl. 424/401; 427/2.14; 424/490; 977/773

(57) ABSTRACT

The invention relates to the filling of hydrophobic or hydrophobically modified porous microparticles with hydrophobic substances with subsequent encapsulation of the resulting hydrophobic particles using layer-by-layer (LbL) polyelectrolyte technology for the purpose of preparing homogeneous suspensions in water, and also for the controlled release of the encapsulated active ingredients. Through a specific modification of the LbL surface it is possible to realize preferred adhesion at the target site.

The invention relates to the filling of hydrophobic or hydrophobically modified porous microparticles with hydrophobic substances with subsequent encapsulation of the resulting hydrophobic particles using layer-by-layer (LbL) polyelectrolyte technology for the purpose of preparing homogeneous suspensions in water, and also for the controlled release of the encapsulated active ingredients. Through a specific modification of the LbL surface it is possible to realize preferred adhesion at the target site.

Microcapsules formed of alternately adsorbed polyelectrolyte layers (layer by layer LbL) are known, for example, from [1] and described in DE 19812083 A1, DE 199 07 552 A1, EP 0 972 563, WO 99/47252 and U.S. Pat. No. 6,479,146, the disclosure content of which is hereby incorporated in its entirety. On account of their adjustable semipermeability, such capsule systems have a high application potential as microreactors, drug delivery systems etc. The prerequisite is filling with appropriate active ingredients, enzymes, polymers or catalysts.

Hitherto, LbL microcapsules have been prepared which can be filled on the inside with macromolecular and water-soluble substances. The technologies known hitherto do not allow sparingly water-soluble or hydrophobic active ingredients to be encapsulated in largely monodisperse form with polyelectrolyte films.

The following options for microencapsulation of hydrophobic active ingredients are known:

a) Complex coacervation
In this process, oil-in-water emulsions of the hydrophobic active ingredient are prepared and provided with a shell of gelatin and gum arabic by changing the pH of the aqueous phase. Such polydisperse capsules can even be dried, but generally only release the active ingredients again following mechanical action or if the pH is changed.
   b) Emulsion polymerization
In emulsions, at the oil/water interface, monomers are crosslinked to give polymeric shells. The dispersity of these capsules is likewise high and is determined by the quality of the emulsions. Also, the release rates cannot be so finely tuned as in the case of LbL layers.
   c) Solubilization of solid active ingredients
According to the Patents WO 01/51196, WO 2004/030649 A2 and PCT/EP03/10630, ground active ingredients are provided with a few LbL layers in order to achieve better stability of the suspensions in aqueous solution. In this method, polydisperse and nonspherical particles are obtained. During the coating, particularly on very extensive crystallites (e.g. needles or platelets), splinters result, which lead to nonuniform release rates and to subsequent instability of the suspension as a consequence of Ostwald ripening or of aggregation of the now partially hydrophobic surfaces.
   d) Core-shell structures
A further process (not prepublished DE 10 2004 013 637.8 dated 19 Mar. 2004 from the same applicant) uses the adsorption of materials in porous particles with subsequent encapsulation via LbL technology. Here, spherical and largely monodisperse particles are obtained in which the release can also be modified via the polyelectrolyte layers. However, with the described hydrophilic particles with a high surface charge it is not possible to immobilize hydrophobic active ingredients or even water-insoluble oils.

It is therefore an object of the present invention to indicate a process for the encapsulation of hydrophobic active ingredients in which the active ingredients:
a) can be enriched in higher concentration in the inside of porous materials
b) the hydrophobic particles can be suspended in water with the help of LbL polyelectrolyte layers
c) the active ingredients can be released in a delayed manner or triggered manner.

According to the invention, this object is achieved by a process for the preparation of active-ingredient-laden particles comprising the following steps:
porous templates with a hydrophobic interior and exterior surface are prepared, the porous templates being porous microparticles with a diameter of less than 100 μm;
at least one hydrophobic active ingredient to be encapsulated is adsorbed in the templates;
the templates with the active ingredient absorbed therein are suspended in an aqueous medium; and
a hydrophilic LbL capsule shell is formed around the porous templates by applying alternately charged polyelectrolyte and/or nanoparticle layers so that a stable suspension of monodisperse particles in the aqueous medium is formed.

The porous templates may be:
synthetic and/or naturally occurring inorganic hydrophilic microparticles whose interior and exterior surface is hydrophobicized; or
hydrophobic organic microparticles with a porous structure.

The LbL coating of the laden templates with hydrophobic surfaces leads to a stable suspension in the aqueous medium without auxiliaries or additives being required.

The templates laden with the absorbed active ingredient can be suspended in the aqueous medium with the help of at least one additive, where the additive stabilizes the particles in the aqueous medium by adsorption to the surface.

Interior surface is understood as meaning the surface formed by the pore walls, whereas exterior surface means the surface of the template facing outwards.

For the purposes of the present invention, porous hydrophilic microparticles are understood in particular as meaning particles of inorganic alumosilicates or pure silicates which have a large number of pores or internal cavities. These pores are hydrophobicized by suitable chemical processes. Alternatively, hydrophobic organic microparticles can also be used without further modification. Filling of the templates advantageously takes place with the help of a solvent in which the hydrophobic active ingredient is readily soluble. After the filling, the very hydrophobic templates are suspended in an aqueous solution with the help of additives, such as, for example, surfactants or amphiphilic polymers. Then, alternating layers of polycation and polyanion are applied in accordance with customary LbL coating technology. Above at least 2 layers, the particles become increasingly electrostatically stabilized, meaning that aggregation phenomena scarcely arise in the suspension, and an addition of surfactants or other auxiliaries is no longer required.

It has proven favourable if, following the suspension of the laden hydrophobic templates with the help of at least one additive, a polyelectrolyte which has the same charge as the additive is added to the aqueous medium. If the additive carries a positive charge, a polycation is added, and in the case of a negatively charged additive, a polyanion. This addition has proven necessary for the structure of the capsule shell.

The capsule shell consists of at least 2 to 3 or more alternately charged polyelectrolyte layers and/or nanoparticle layers. Capsule shells with up to 20 or 30 such alternately charged layers are possible. The individual layers are applied one after the other, the polyelectrolytes and/or nanoparticles used for the structure assembling electrostatically on the previously applied layer.

The templates used are porous microparticles whose size is preferably less than 100 μm. The microparticles have pores with, for example, a pore width of 0.3 nm-100 nm, preferably of 1 nm-30 nm. In many applications, the lower limit of the pore width can be between 1 nm and 6 nm, for example 2 nm or 4 nm, and the upper limit of the pore width can be between 10 nm and 40 nm, for example 15 nm or 30 nm. In principle, the pore width should be sufficiently large to allow the active ingredients to be encapsulated to penetrate into the pores and be deposited in the pores. Preference is therefore given to porous templates (hydrophobic and/or hydrophobically modified hydrophilic microparticles) with a large interior surface, the interior surface being formed by the inner walls of the pores.

When using hydrophilic inorganic microparticles, such as, for example, silicates or alumosilicates, the interior and exterior surface of the particles is hydrophobically modified prior to loading. Besides other chemical processes, the reaction of the Si—OH groups with alkyl- or aryl-alkoxysilanes in particular is suitable for this. The degree of hydrophobicity can be selectively adjusted via the length and the number of alkyl chains per surface segment. Consequently, the energy of interaction between porous particle and hydrophobic active ingredient can be adjusted, which permits control of the degree of filling with the hydrophobic material and, above all, the release rate.

Following construction of the capsule shell, the templates can be suitably detached so that only the active ingredient remains enclosed in the capsule shell.

Furthermore, the object is achieved by particles with:
a diameter of less than 100 μm;
a porous core with a hydrophobic interior and exterior surface in which at least one hydrophobic active ingredient is adsorbed; and
a capsule shell of a plurality of layers of alternately charged polyelectrolyte and/or nanoparticle layers.

The porous core is the described porous templates. Optionally, between the porous core and the capsule shell can be arranged a primer layer which surrounds the core and contributes to the improvement of the construction of the capsule shell.

The particles prepared and filled with the active ingredient can advantageously be used in many areas, for example:
for the encapsulation, for the adhesion to the desired target site and for the release of hydrophobic active ingredients in the pharmaceutical and cosmetics industry;
for the encapsulation of oils and fragrances as stable aqueous suspensions in the fields of cosmetics, pharmacy, detergent industry and care product industry (leather industry);
for the encapsulation of oils and hydrophobic solids with a defined release rate following adhesion to desired surfaces;
for the encapsulation of oils and hydrophobic solids with triggered release upon changing the ambient conditions (pH, mechanical stress, influence of solvent or surfactant, drying, heat etc.);
for applications in the food industry and agriculture and forestry.

Further advantageous embodiments of the invention, independently of whether the process or the filled particles are concerned, are described below by reference to the figures. In these:

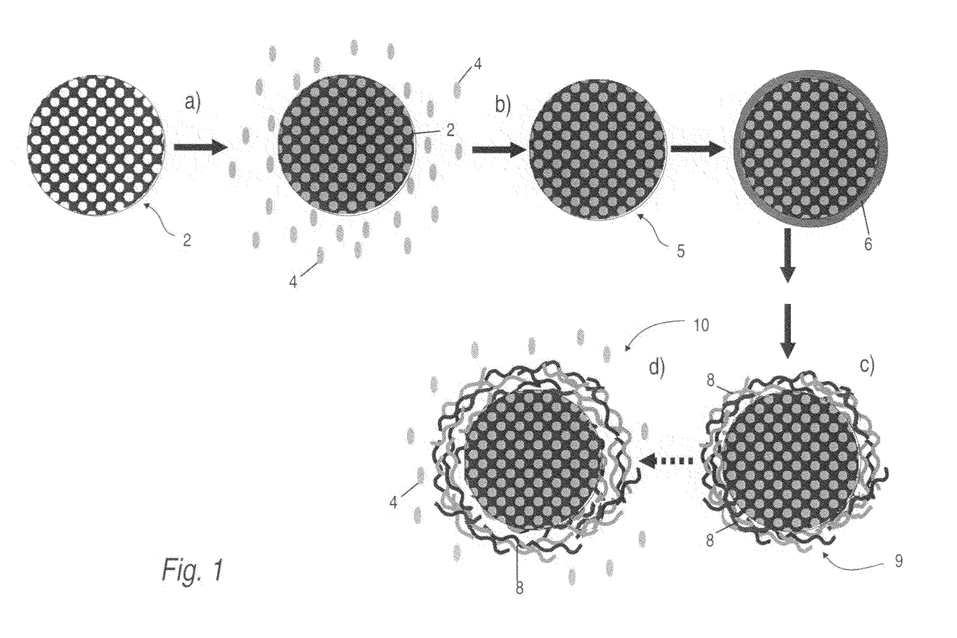

FIG. 1 shows individual process steps of the process according to the invention and also the resulting microcapsules filled with hydrophobic materials;

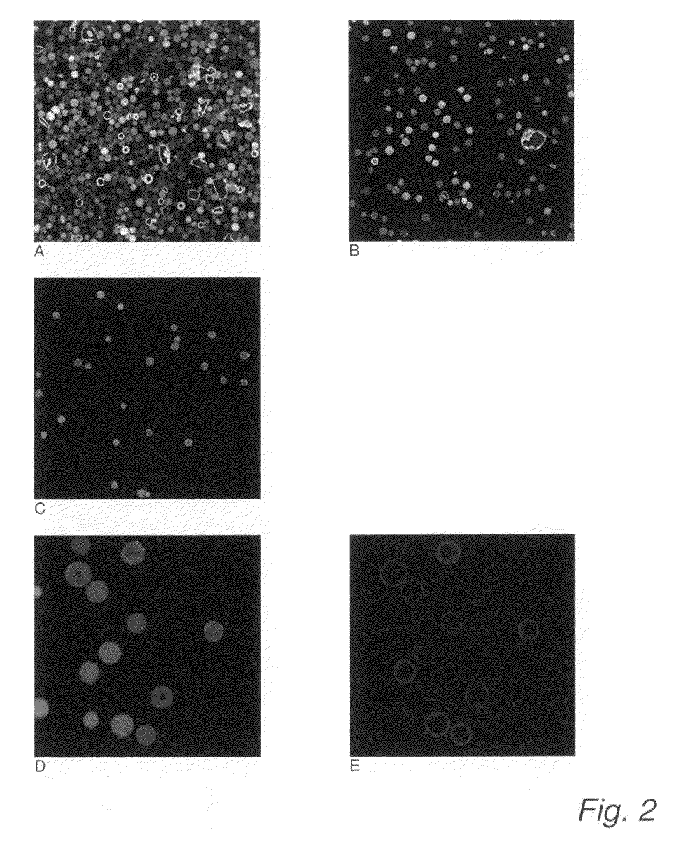

FIG. 2a shows confocal images of templates loaded with a hydrophobic active ingredient which are suspended in water with an auxiliary;

FIG. 2b shows loaded templates with a capsule shell formed of two LbL layers;

FIG. 2c shows loaded templates with a capsule shell formed of six LbL layers;

FIG. 2d shows confocal micrograph of the fluorescent active ingredient in the interior of the particles;

FIG. 2e shows confocal micrograph of the fluorescent (Cy5 labelled) LbL capsule shell;

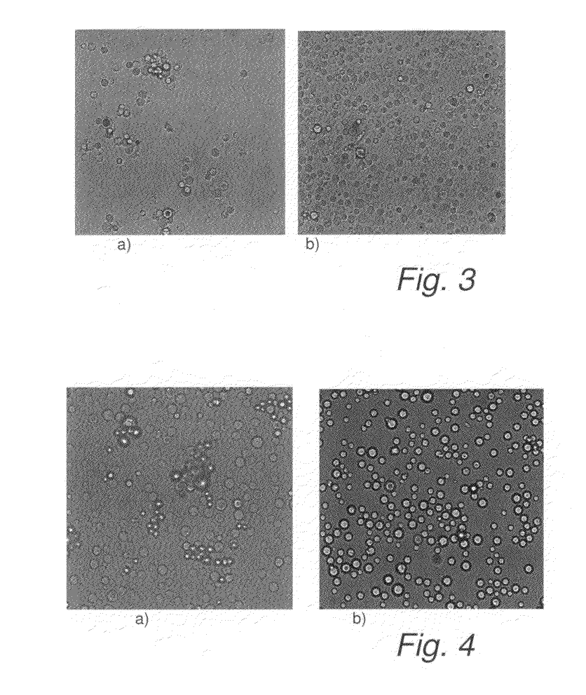

FIG. 3 shows transmission micrographs of imidacloprid-filled particles a) after one PSS layer b) after 6 layers of PAH/PSS (separate imidacloprid crystals are sometimes also to be found outside the capsules);

FIG. 4 shows transmission micrographs of 5 μm particles filled with peppermint oil a) after one PSS layer b) after 4 further layers of PAH/PSS;

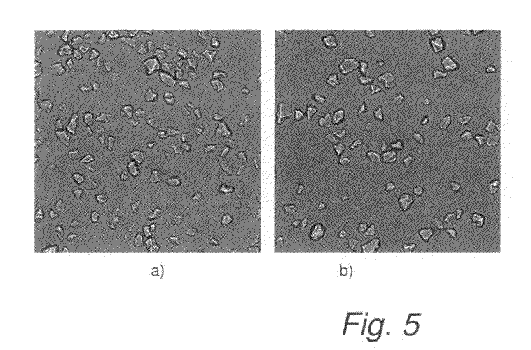

FIG. 5 shows OMC filled particles a) with 6 polyelectrolyte layers (positively charged) and b) with 7 polyelectrolyte layers (negatively charged).

The individual process steps are explained by reference to FIG. 1. Preferably, colloidal hydrophilic microparticles (templates) with a defined porosity are used whose interior and exterior surface is hydrophobically modified with, for example, alkylalkoxysilanes. These microparticles are filled with the materials to be encapsulated (called active ingredient below) in the desired concentration. FIG. 1 shows the filling with an active ingredient which at a later point in time is immobilized permanently in the interior or, in the case of appropriate wall permeability, is released in a metered manner.

The active ingredient can be any liquid or solid hydrophobic material made of inorganic or organic material. In particular, the active ingredients to be encapsulated are solids or oils which cannot be dissolved in water, or can be dissolved only with very great difficulty. Usually, further auxiliaries (e.g. surfactants) are required to produce stable suspensions or emulsions. In particular, the substances to be encapsulated may be pharmaceutical or cosmetic active ingredients, such as fragrances, skin protection oils and fats, UV absorbers, and/or washing and care composition additives, such as lipids, silicone oils and/or lubricants and/or crop protection compositions. The active ingredients to be encapsulated can have a different affinity or binding constant with regard to the deposition in the pores. The active ingredients occupy the available binding sites on the interior surface depending on their binding constants. The interaction to the surfaces can be adapted through the degree of hydrophobicization (number and size of alky or aryl groups).

### Filling the Templates (Step A)

Filling of the hydrophobicized porous templates 2 with hydrophobic active ingredients 4 takes place through attractive interaction based predominantly on dispersion interactions (also called hydrophobic interactions or van der Waals interactions). In particular, two different processes are used for the filling:
1. The hydrophobic material 4 is dissolved in an organic, water-miscible solvent, such as, for example, ethanol, acetone, acetonitrile etc. Following the addition of the particles 2, the polarity of the solvent is increased through stepwise addition of water to the point of saturation of the active ingredient in the solution with constant stirring. The supernatant is washed away with water.
2. The active ingredient 4 is dissolved in an organic solvent in the amount to be used for filling. Following the addition of the particles 2, the solvent is completely evaporated with stirring and optionally with heating or under reduced pressure. The filled particles 5 remain. Only in the case of an excessively high amount of active ingredient or if the affinity of the active ingredient to the particle surface is too low are remains of the active ingredient in crystalline or oily form left behind outside of the particles. Otherwise, the active ingredient is located exclusively within the particles.

For the filling with the active ingredients, pore sizes are used which are matched to the size of the molecules to be filled. Especially in the case of porous silicate particles, molecules between 0.1 and 5000 kDa (100 g/mol-5,000,000 g/mol) can be intercalated in pore sizes of from 0.4 to 100 nm. A plurality of active ingredients can also be intercalated, in the case of comparable binding constants, simultaneously, or, in the case of different binding constants, sequentially. In this connection, the active ingredient with the higher binding constant is not completely filled, i.e. its concentration is chosen such that this active ingredient does not occupy all of the available binding sites. Afterwards, the incompletely filled particles are filled with the further active ingredient. As a result, the templates 2 are largely filled with the active ingredient(s) 4.

### Solubilization (Step B)

The now filled templates 5 are coated in an aqueous solution by the known LbL process or similar single-step processes. For this, in the first step, they have to be suspended in water, which in most cases is only possible with the addition of surfactants of various types or similar amphiphilic polymers. Here, suitable auxiliaries are to be chosen in the lowest possible concentration which do not dissolve the active ingredient out of the particle interior and solubilize it in the form of micells. In addition, following suspension in water with the help of the surfactant, a polyelectrolyte with the same charge as the surfactant can be added. Although the mode of action is not completely clear, and without wishing to impose any limitation, it is assumed that this polyelectrolyte, which is also referred to as primer electrolyte, partially suppresses the surfactant and/or together with the surfactant forms a primer layer.

Optionally, a primer layer 6 can also be applied. The optional primer layer 6 is not described in the further steps.

### Coating (Step C)

The filled particles suspended in water are coated with alternately cationically and anionically charged substances (polyelectrolytes), preferably polymers. Even after one polyelectrolyte layer, the surfactant can be largely dispensed with. Depending on the degree of charging and hydrophobicity, after 2-6 layers, homogeneous particle suspensions in water are obtained in which the particles are present in electrostatically stabilized form (without a tendency towards aggregation). Further additives for preparing stable suspensions are no longer required.

The permeability of the LbL capsules can be specifically adjusted for the particular encapsulated material through the number of applied layers, the polyelectrolyte combination, through an after treatment by means of annealing, or by implementation of further substances into the capsule wall^{[8]}. Following construction of the capsule wall, coated particles 10 with a filled porous core are present. Suitable substances for forming the capsule wall and suitable process courses can be found in the already mentioned documents DE 198 12 083 A1, DE 199 07 552 A1, EP 0 972 563, WO 99/47252 and U.S. Pat. No. 6,479,146.

The adsorption of these particles as defined targets can be achieved, sometimes very specifically, via interactions between the outermost polyelectrolyte layer and the target. For this serves, in the simplest case, the adjustment of the external charge of the particles (negative or positive), or more defined the coating with polyelectrolytes which have a high affinity to the desired surfaces or, quite specifically, via the recognition of receptors via biomolecules covalently coupled to the particle surface (streptavidin, biotin, proteins, peptides, DNA, RNA).

### Optional Release of the Active Ingredient (Step D)

The active ingredients can optionally be released in a controlled manner from the microparticles 10 filled with active ingredient (FIG. 1). The release can take place here either in a delayed manner, or else triggered by a signal. Such a triggering can be achieved, for example, by:
a) drying the particles
b) increased concentration of (organic solvent) which readily dissolves the active ingredient
c) surfactants which solubilize the active ingredient
d) pH changes
e) mechanical stress
f) increased temperature
g) and a combination of the preceding factors.

The release rate can be varied through various parameters, such as, for example:
1. binding constant (hydrophobicity) to the particle surface
2. thickness of the LbL capsule wall
3. capsule wall material
4. crosslinking of the wall material

### EXAMPLES

### 1. Hydrophobic Dye Perylene:

12.5 mg of the model dye perylene were dissolved in 6 ml of chloroform. A suspension of 100 mg of spherical, porous silica templates (diameter 5 μm, pore size 6 nm) in 1 ml of chloroform was added thereto. The surface of the templates is hydrophobically modified with C18 alkyl chains (RP 18, chromatography material). The solvent was evaporated at 40° C. in a drying cabinet with stirring. The powder which remained was resuspended in water with the help of 1% sodium dodecyl sulphate (SDS). FIG. 2a shows a confocal micrograph of the particles filled with perylene which, despite the SDS, are still present in very aggregated form. Besides the fluorescence of the perylene in the particles, a few larger (and lighter) crystals of the pure dye can also be seen which no longer arise when using smaller fill amounts. The SDS solution was centrifuged off and the particles were incubated with 500 μl of a solution of 1 g/l Cy5 labelled poly(allylamine hydrochloride) (PAH-Cy5, MW 70000 g/mol), pH 6.5 and 0.5 M NaCl for 20 min with brief application of ultrasound. After washing the particles three times with water, 500 μl of a solution of 1 g/l of poly(sodium 4-styrenesulphonate) (PSS, MW 70000 g/mol) in 0.5 M NaCl with pH 6.1 are added. The suspension was incubated for 20 min with brief application of ultrasound, and the supernatant was removed by washing three times with water. After these two LbL layers, aggregation still occurred here and there (FIG. 2b). Subsequent coating reduced the aggregation tendency yet further, until from the 6^{th} layer onwards all of the particles were present in individually separated form in water (FIG. 2c). In the enlargement, the green fluorescent hydrophobic perylene can clearly be seen in the interior (FIG. 2d). The long-wave (shown here in blue) fluorescent, Cy5 labelled, hydrophilic capsule wall (FIG. 2e) forms a closed layer around the particle and does not penetrate into the hydrophobic interior.

Analysis of the particles obtained revealed, in the interior, a concentration of 8.5 mg of perylene per 100 mg of particles (FIG. 2d). The concentration of the perylene in the interior of the particles was determined via the fluorescence by confocal microscopy by reference to comparison solutions. Here, the high concentration of dye in the interior of the capsules can lead to self-quenching, which feigns a lower concentration.

### 2. Hydrophobic Active Ingredient Imidacloprid:

25 mg of the somewhat water-soluble active ingredient imidacloprid were dissolved in 3 ml of acetone, and 100 mg of spherical, porous silica templates (diameter 5 μm, pore size 6 nm) were added. After incubation for 30 min, water was added stepwise until the solution reached saturation of imidacloprid, evident from further considerable cloudiness. The supernatant was centrifuged off and the particles were suspended with 1% SDS solution. In this connection, here and in all of the following washing and coating solutions, imidacloprid saturated solutions were used in order to prevent a dissolving out from the particles. After the coating with PSS and PAH (analogously to Example 1), separate particles filled with 15% m/m (mass of imidacloprid/mass of particles) were obtained (FIG. 3). The amount of imidacloprid was determined by releasing the active ingredient in a large excess in methanol with subsequent UV/vis spectroscopic analysis of the supernatant.

### 3. Perfume Oil (Orange Oil and Peppermint Oil)

50 mg of peppermint oil were dissolved in 1 ml of acetone. A suspension of 200 mg of spherical, porous silica templates (diameter 5 μm, pore size 6 nm, hydrophobic C18 modification) suspended in 2 ml of acetone was added thereto. The solvent was evaporated at 20° C. with stirring. The powder which was left behind was resuspended with the help of 1% sodium dodecyl sulphate (SDS) in 2.5 ml of water with ultrasound. The SDS solution was centrifuged off and the particles were incubated with 2.5 ml of a solution of 1 g/l of PSS at pH 5.6 and 0.5 M NaCl for 20 min with brief application of ultrasound. After washing the particles three times with water, the particles are again significantly aggregated (FIG. 4a). Coating with a further 4 layers of PAH and PSS produced well separated particles in water (FIG. 4b). Even after storage for several weeks, they can be shaken up again without problems. Incubation of the particles for two hours in methanol produced a released amount of peppermint oil of 22% per particle. This amount was determined in the supernatant after centrifuging off the particles by means of UV spectroscopy at 230 nm. The orange oil was encapsulated identically and produced a released amount of 19.2%.

While the release in methanol takes place quantitatively, in water virtually no perfume oil is washed out of the particles, i.e. the suspensions are virtually odourless. However, a triggered release important, for example, for cosmetic or textile applications, can be achieved by drying or heating the particles or by mechanically destroying them. For this, a suspension of positively charged peppermint particles was placed on cotton, which adhere well to the negatively charged textile surface. As long as the material is wet, the samples are virtually odourless. After drying, the odour intensifies and remains constant over a long period. If the substance is rubbed or heated (2 min at 70° C.), the samples smell more intense, with the odour subsiding again with time. Analysis of the odours was carried out independently by 4 subjects.

### 4. Photoabsorber OMC/Nonspherical Particles

The hydrophobic octyl methoxycinnamate oil (OMC) used in cosmetics as UVA absorber was encapsulated in nonspherical silicate particles. 380 mg of OMC were dissolved in 5 ml of acetone. A suspension of 1.5 g of broken, porous silica templates (size about 5 μm, pore size 10 nm, hydrophobic C18 modification) suspended in 2 ml of acetone was added thereto. Such particles are significantly less expensive than spherical particles but have the disadvantage that, on account of the relatively large contact areas, they form aggregates to a greater extent than do the spherical alternatives. The solvent was evaporated at 20° C. with stirring. The powder which was left behind was resuspended with the help of 1% sodium dodecyl sulphate (SDS) in 2.5 ml of water with ultrasound. The SDS solution was centrifuged off and the particles were incubated with 2.5 ml of a solution of 1 g/l of sodium alginate at pH 5.6 and 0.5 M NaCl for 20 min with brief application of ultrasound. Coating with a further 5 (exterior positive) and 6 layers (exterior negative) of PAH and PSS produced, for both charges, well separated particles in water (FIG. 5a, b). Incubation of the particles for 2 hours in methanol produced a released amount of OMC of 22% per particle. This amount was determined after the particles had been centrifuged off by means of UV spectroscopy at 310 nm in the supernatant.

Release in water does not take place on account of the insolubility of the OMC. By contrast, if the aqueous suspension is added to methanol, then the OMC is released (triggered) within seconds. It is well known that LbL layers in nonaqeuous solvents greatly increase their permeability as a result of structural changes.^{[2]}. Decreasing concentration of methanol in water leads to an increasingly slower and incomplete release (dependent on the Nernst distribution coefficients between the particle phase and the solution phase) of the oil.

### LIST OF REFERENCES

2 porous template
4 active ingredient
5 template filled with active ingredient
6 primer layer
8 layers of the capsule shell
9 capsule shell
10 filled, hydrophilic particles

### LITERATURE

[1] E. Donath, G. B. Sukhorukov, F. Caruso, S. A. Davis, M. Möhwald, Angewandte Chemie-International Edition 1998, 37, 2202-2205.
[2] B.-S- Kim, O. Lebedeva, O. I. Vinogradova et al. Macromolecules 2005, 38, 5214-5222

What is claimed is:

1. A process for the preparation of active-ingredient-laden particles, comprising the following steps:
preparing porous templates having a hydrophobic interior and exterior surface, the porous templates being porous microparticles with a diameter of less than 100 μm;
adsorbing at least one hydrophobic active ingredient to be encapsulated into the porous templates;
suspending the templates with the active ingredient absorbed therein in an aqueous medium; and
forming a hydrophilic layer-by-layer (LbL) capsule shell around the porous templates by applying alternately charged polyelectrolyte and/or nanoparticle layers so that a stable suspension of monodisperse particles in the aqueous medium is formed.

2. The process according to claim 1, characterized in that the porous templates are porous synthetic and/or naturally occurring inorganic hydrophilic microparticles and, wherein the interior and exterior surfaces of the hydrophilic microparticles are hydrophobicized to form porous templates having a hydrophobic exterior surface and a hydrophobic interior surface.

3. The process according to claim 2, characterized in that the hydrophilic microparticles are silicates or alumosilicates.

4. The process according to claim 1, characterized in that the templates are porous hydrophobic organic microparticles.

5. The process according to claim 1, characterized in that the hydrophobic active ingredient to be encapsulated is a liquid or a solid.

6. The process according to claim 1, characterized in that, the aqueous medium further comprises one or more additives to stabilize the porous templates in the aqueous medium by adsorption to the surface.

7. The process according to claim 6, characterized in that, following the suspension of the hydrophobic templates with the active ingredient absorbed therein, a primer polyelectrolyte of equal charge to the additive is added to the aqueous medium.

8. The process according to claim 7, characterized in that, following the addition of the primer polyelectrolyte, the capsule shell is applied to the template by the addition of a polyelectrolyte and/or nanoparticle of opposite charge to the primer polyelectrolyte.

9. The process according to claim 6, characterized in that the additive is an amphiphilic polymer.

10. The process according to claim 1, characterized in that the surface of the LbL capsule shell further comprises selectively binding ligands.

11. A particle comprising
a diameter of less than 100 μm;
a porous core with a hydrophobic interior and exterior surface, wherein at least one hydrophobic active ingredient is adsorbed in the pores of the porous core; and
a hydrophilic capsule shell around the porous core comprising a plurality of layers of alternately charged polyelectrolyte and/or nanoparticle layers.

12. The particle according to claim 11, characterized in that the porous core is comprised of hydrophobic organic materials.

13. The particle according to claim 11, characterized in that the porous core consists of a hydrophilic material having a hydrophobicized surface.

14. The particle according to claim 13, characterized in that the porous core consists of silicates or alumosilicates.

15. The particle according to claim 11, characterized in that the pores of the porous core have a pore width of 0.3 nm-100 nm.

16. (canceled)

17. The process according to claim 1, wherein the active ingredient is selected from the group consisting of oils, hydrophobic pharmaceuticals, and cosmetics.

18. The particle according to claim 11, wherein the active ingredient is selected from the group consisting of oils, hydrophobic pharmaceuticals, and cosmetics.

19. The particle according to claim 15, characterized in that the pores of the porous core have a pore width of 1 nm to 30 nm.
